(19) United States
(12) Patent Application Publication
(10) Pub. No.: US 2024/0279194 A1
(43) Pub. Date: Aug. 22, 2024

(54) SHUTDOWN METHOD FOR A PROCESS FOR PREPARING AN OLEFIN OXIDE

(71) Applicants: BASF SE, Ludwigshafen am Rhein (DE); Dow Global Technologies LLC, Midland, MI (US)

(72) Inventors: Dominic RIEDEL, Ludwigshafen am Rhein (DE); Maria Angel SAN PIO BORDEJE, Ludwigshafen am Rhein (DE); Joaquim Henrique TELES, Ludwigshafen am Rhein (DE); Andrei-Nicolae PARVULESCU, Ludwigshafen am Rhein (DE); Dylan SEGERS, Antwerp (BE); Meinolf WEIDENBACH, Stade (DE); Franciscus Johannes Robertus VAN NEER, Hoek (NL); Massimo BERGAMO, Stade (DE)

(21) Appl. No.: 18/569,645

(22) PCT Filed: Jun. 14, 2022
(86) PCT No.: PCT/EP2022/066154

§ 371 Date: Dec. 13, 2023

(30) Foreign Application Priority Data
Jun. 15, 2021 (EP) 21179509.1

(51) Int. Cl. C07D 303/04

(52) U.S. Cl. CPC C07D 303/04

(57) ABSTRACT

In a first aspect, the present invention relates to a shutdown method for a process for preparing an olefin oxide comprising a normal run stage, wherein the normal run stage comprises providing olefin, hydrogen peroxide and organic solvent into an epoxidation zone comprising an heterogeneous epoxidation catalyst, so that a reaction mixture comprising olefin, hydrogen peroxide and organic solvent is formed and subjecting the reaction mixture to epoxidation reaction conditions in the epoxidation zone, thereby obtaining a mixture comprising olefin oxide and organic solvent; wherein the shutdown method comprises (a) providing olefin and organic solvent for a period of time t1 to the epoxidation zone, so that a mixture is formed, which is essentially free of hydrogen peroxide; (b) contacting the mixture from (a) over t1 under epoxidation reaction conditions in the epoxidation zone with the heterogeneous epoxidation catalyst; (c) removing during t1 an effluent stream from the epoxidation zone, the effluent stream comprising olefin and organic solvent. In a second aspect, the present invention relates to a process for preparing an olefin oxide comprising a normal run stage and a shutdown stage, wherein the normal run stage comprises (A) providing olefin, hydrogen peroxide, water and organic solvent into an epoxidation zone comprising an heterogeneous epoxidation catalyst, so that a reaction mixture comprising olefin, hydrogen peroxide, water and organic solvent is formed; (B) subjecting the reaction mixture from (I) to epoxidation reaction conditions in the epoxidation zone, thereby obtaining a mixture comprising olefin oxide, water and organic solvent; (C) removing an effluent stream from the epoxidation zone, comprising olefin oxide, water and organic solvent; wherein the shutdown stage comprises steps (a), (b) and (c) as described with respect to the first aspect.

In a first aspect, the present invention relates to a shutdown method for a process for preparing an olefin oxide comprising a normal run stage, wherein the normal run stage comprises providing olefin, hydrogen peroxide and organic solvent into an epoxidation zone comprising an heterogeneous epoxidation catalyst, so that a reaction mixture comprising olefin, hydrogen peroxide and organic solvent is formed and subjecting the reaction mixture to epoxidation reaction conditions in the epoxidation zone, thereby obtaining a mixture comprising olefin oxide and organic solvent;
wherein the shutdown method comprises
   (a) providing olefin and organic solvent for a period of time t₁ to the epoxidation zone, so that a mixture is formed, which is essentially free of hydrogen peroxide;
   (b) contacting the mixture from (a) over t₁ under epoxidation reaction conditions in the epoxidation zone with the heterogeneous epoxidation catalyst;
   (c) removing during t₁ an effluent stream from the epoxidation zone, the effluent stream comprising olefin and organic solvent.

In a second aspect, the present invention relates to a process for preparing an olefin oxide comprising a normal run stage and a shutdown stage, wherein the normal run stage comprises
(A) providing olefin, hydrogen peroxide, water and organic solvent into an epoxidation zone comprising an heterogeneous epoxidation catalyst, so that a reaction mixture comprising olefin, hydrogen peroxide, water and organic solvent is formed;
(B) subjecting the reaction mixture from (A) to epoxidation reaction conditions in the epoxidation zone, thereby obtaining a mixture comprising olefin oxide, water and organic solvent;
(C) removing an effluent stream from the epoxidation zone, comprising olefin oxide, water and organic solvent;
wherein the shutdown stage comprises steps (a), (b) and (c) as described with respect to the first aspect.

Olefin oxides such as propylene oxide are important intermediates in the chemical industry. A suitable process for the preparation of olefin oxide starts from the respective olefin and makes use of hydrogen peroxide as oxidizing agent, organic solvents, water and heterogeneous epoxidation catalysts such as titanium containing zeolites. Due to the importance for industrial-scale processes, it is desired to carry out such epoxidation reactions as efficiently as possible.

Any process and even more important each industrial scale process comprises at least three stages, that is a start-up stage, wherein the reaction is started, a normal run stage, wherein the reaction is carried out so that the desired product is obtained, and finally a shutdown stage, where the reaction is terminated and the reaction vessel may be emptied, for example, in order to enable catalyst regeneration, replacement etc. . . . Especially when, as it is often the case, epoxidations are carried out in several reactors in parallel, shutdown is of importance, since normally, any epoxidation process is part of a highly integrated system, which requires work-up of almost all streams as well as recycling of reactands, solvents etc. coming from these reactors. Even if only one reactor is used, it is mandatory for any industrial plant to recover and recycle unconverted materials such as the olefin and the reaction solvent. Thus, care has to be taken regarding the components present in any stream coming from a reactor. In case one of a plurality of reactors has to be shutdown, while one or more of the others remain in operation, it is mandatory that the streams coming from this reactor do not negatively impair the overall balance and also do not cause safety issues. However, it is known that simply cutting off all feed streams at once is detrimental in view of, for example, residues, which remain within the reactor. CN 102260226 A discloses a process for the epoxidation of 3-chloropropene with hydrogen peroxide in methanol using a TS-1 epoxidation catalyst with focus on regeneration of spent catalyst. After a certain period of epoxidation time, the feeds of 3-chloropropene and hydrogen peroxide are both simultaneously stopped, while methanol is still fed to the epoxidation reactor for washing the catalyst.

It was an object of the present invention to provide an advantageous method for shutting down an epoxidation reaction stepwise.

The present invention thus relates to a shutdown method for a process for preparing an olefin oxide comprising a normal run stage, wherein the normal run stage comprises providing olefin, hydrogen peroxide and organic solvent into an epoxidation zone comprising an heterogeneous epoxidation catalyst, so that a reaction mixture comprising olefin, hydrogen peroxide and organic solvent is formed and subjecting the reaction mixture to epoxidation reaction conditions in the epoxidation zone, thereby obtaining a mixture comprising olefin oxide and organic solvent; wherein the shutdown method comprises
(a) providing olefin and organic solvent for a period of time t₁ to the epoxidation zone, so that a mixture is formed, which is essentially free of hydrogen peroxide;
(b) contacting the mixture from (a) over t₁ under epoxidation reaction conditions in the epoxidation zone with the heterogeneous epoxidation catalyst;
(c) removing during t₁ an effluent stream from the epoxidation zone, the effluent stream comprising olefin and organic solvent.

It was found that terminating first the feed of aqueous hydrogen peroxide, i.e. using in the beginning of the shutdown a mixture comprising olefin and organic solvent, clearly resulted in much lower oxygen concentration in the effluent stream over the complete shutdown stage and also during any epoxidation after restart. It could be seen that the O₂ content reached a value which was not more than 50% higher than the O₂ content in the normal run; preferably, the O₂ value decreased compared to the O₂ content in the normal run, and the O₂ content preferably reached a value of less than 100% of the O₂ content in the normal run during the restart. On the other hand, it could be shown that terminating in the beginning of the shutdown the olefin feed first, while maintaining the feed of aqueous hydrogen peroxide solution, i.e. using a mixture comprising aqueous hydrogen peroxide solution and olefin, results in a significant increase of oxygen formation in the effluent stream. Such an effect in turn results in issues in the olefin recovery section of any industrial plant and also causes safety concerns.

“Essentially free of hydrogen peroxide” regarding the mixture in (a) means that said mixture comprises less than 0.2 weight-%, preferably less than 0.1 weight-%, more preferably less than 0.05 weight-%, of hydrogen peroxide, based on the total weight of the mixture. In some preferred embodiments, the molar amount of olefin provided in (a) of the shut-down stage is in the range of 0.25×[(molar amount of olefin provided in the normal-run stage) minus (molar amount of hydrogen peroxide provided in the normal-run stage)] to 3.0×[(molar amount of olefin provided in the normal-run stage) minus (molar amount of hydrogen peroxide the normal-run stage)], more preferably in the range of 0.5×[molar amount of olefin provided in the normal-run stage) minus (molar amount of hydrogen peroxide provided in the normal-run stage)] to 2.5×[(molar amount of olefin provided in the normal-run stage) minus (molar amount of hydrogen peroxide provided in the normal-run stage)], more preferably in the range of 1.8×[molar amount of olefin provided in the normal-run stage) minus (molar amount of hydrogen peroxide provided in the normal-run stage)] to 2.0×[(molar amount of olefin provided in the normal-run stage) minus (molar amount of hydrogen peroxide provided in the normal-run stage)]. Preferably, the weight ratio of organic solvent:olefin (w/w) in the mixture from (a) is in the range of from 18:1 to 1:0.1, preferably in the range of from 15:1 to 1:1, more preferably in the range of from 12:1 to 7:1.

According to a preferred embodiment of the shutdown method, the shutdown stage further comprises
(d) providing organic solvent for a period of time t₂ to the epoxidation zone, wherein the organic solvent is essentially free of hydrogen peroxide and of olefin;
(e) contacting the organic solvent from (d) over t₂ under epoxidation reaction conditions in the epoxidation zone with the heterogeneous epoxidation catalyst,
(f) removing during t₂ an effluent stream from the epoxidation zone, the effluent stream comprising organic solvent.

“Essentially free of hydrogen peroxide and of olefin” regarding the organic solvent from (d) means that said organic solvent comprises less than 0.2 weight-%, preferably less than 0.1 weight-%, more preferably less than 0.05 weight-%, of hydrogen peroxide and less than 1 weight-%, preferably less than 0.5 weight-%, more preferably less than 0.1 weight-%, of olefin, each based on the total weight of the organic solvent.

According to a preferred embodiment of the shutdown method, the shutdown stage further comprises
(g) providing a gaseous stream and passing said gaseous stream for a period of time t₃ into the epoxidation zone comprising the heterogeneous epoxidation catalyst and removing during t₃ the gaseous stream from the epoxidation zone.

Preferably, in step (g), the gaseous stream is provided with a gas flow in the range of from 1500 to 6000 kg/h, more preferably with a gas flow in the range of from 2000 to 5000 kg/h, more preferably with a gas flow in the range of from 2200 to 4500 kg/h, more preferably with a gas flow in the range of from 2500 to 4000 kg/h.

According to a preferred embodiment of the shutdown method, the gaseous stream used in (g) comprises at least 90 volume-%, more preferably at least 95 volume-%, more preferably at least 98 volume-% of nitrogen, based on the total volume of the gaseous stream.

In some embodiments, lean air is used. Lean air is an oxygen-nitrogen mixtures with an oxygen content of up to 10 volume-%, preferably in the range of from 0.1 to 5 volume-%, more preferably in the range of from 0.1 to 2 volume-%, the remainder up to 100 volume-% being nitrogen.

According to a preferred embodiment of the shutdown method, at least one of (a), (b) and (c) preferably (a), (b) and (c), are carried out continuously and/or wherein at least one of (d), (e) and (f) preferably (d), (e) and (f) are carried out continuously and/or wherein (g) is carried out continuously; wherein more preferably at least (a), (b), (c), (d), (e) and (f) are carried out continuously, wherein more preferably (a), (b), (c), (d), (e), (f) and (g) are carried out continuously.

According to a preferred embodiment of the shutdown method, the normal run stage is carried out in continuous mode.

According to a preferred embodiment of the shutdown method, step (a) comprises
(a.1) providing olefin and organic solvent,
(a.2) combining olefin and organic solvent so that a mixture comprising olefin and organic solvent is formed, which is essentially free of hydrogen peroxide;
(a.3) feeding the mixture obtained in (a.2) for a period of time t₁ to the epoxidation zone.

According to a preferred embodiment of the shutdown method, t₁ is a period of time in the range of from 1 minute to 10 hours, preferably in the range of from 10 minutes to 5 hours, more preferably in the range of from 30 minutes to 2 hours. According to a preferred embodiment of the shutdown method, t₂ is a period of time in the range of from 1 minute to 10 hours, preferably in the range of from 10 minutes to 5 hours, more preferably in the range of from 30 minutes to 2 hours. According to a preferred embodiment of the shutdown method, t₃ is a period of time in the range of from 1 to 48 hours, preferably in the range of from 10 to 36 hours, more preferably in the range of from 20 to 30 hours.

### Epoxidation Conditions

According to a preferred embodiment of the shutdown method, the contacting under epoxidation reaction conditions in the epoxidation zone with the heterogeneous epoxidation catalyst in (b) and/or (f) is carried out at an absolute pressure in the epoxidation zone in the range of from 0.5 to 5.0 MPa, preferably in the range of from 1.5 to 3.0 MPa, more preferably in the range of from 1.8 to 2.8 MPa. Preferably during at least steps (b) and (f), more preferably during all steps (a) to (f), the pressure in the epoxidation zone is maintained in the range of from 0.5 to 5.0 MPa, preferably in the range of from 1.5 to 3.0 MPa, more preferably in the range of from 1.8 to 2.8 MPa, optionally by adding an inert gas, preferably selected from the group consisting of helium, neon, argon, krypton, radon, xenon, nitrogen and mixtures of two or more of these inert gases, more preferably nitrogen (N₂).

According to a preferred embodiment of the shutdown method, the contacting under epoxidation reaction conditions in the epoxidation zone with the heterogeneous epoxidation catalyst in (b) and/or (f) is carried out at a temperature in the epoxidation zone in the range of from 20 to 75° C., preferably in the range of from 22 to 75° C., more preferably in the range of from 24 to 70° C., more preferably in the range of from 25 to 65° C. Preferably during at least steps (b) and (f), more preferably during all steps (a) to (f), the temperature in the epoxidation zone is kept within the above-mentioned temperature range. The temperature in the epoxidation zone in the context of this application is defined as the entrance temperature of the cooling medium to the mantle of the reactor. In case there is more than one entrance or even more than one reaction zone each with a separate entrance for the cooling medium, then the temperature in the reaction zone will be defined as the weight averaged temperature of all the cooling medium feeding streams. The reactor is preferably at least one, preferably continuously operated, reactor such as a tube reactor or a tube bundle reactor which preferably contains at least one cooling jacket surrounding the at least one tube. A cooling medium flows through the cooling jacket. The nature of the cooling medium is not particular restricted as long as it is sufficient for adjusting the temperature in the epoxidation zone. For example, the cooling medium comprises water, wherein it may additionally comprise additives such as aliphatic C2 to C5 mono-alcohols, aliphatic C2 to C5 diols and mixtures of two or more thereof. Preferably, ≥90 weight-%, more preferred ≥95 weight-% of the cooling medium are water, based on the total weight of the cooling medium.

Preferably, step (g) comprises
(g.1) providing a gaseous stream and passing said gaseous stream for a period of time t_{3.1} into the epoxidation zone comprising the heterogeneous epoxidation catalyst and removing during t_{3.1} the gaseous stream from the epoxidation zone, wherein the pressure in the epoxidation zone is in the range of from 0.5 to 5.0 MPa, preferably in the range of from 1.5 to 3.0 MPa, more preferably in the range of from 1.8 to 2.8 MPa;
(g.2) providing a gaseous stream and passing said gaseous stream for a period of time t_{3.2} into the epoxidation zone comprising the heterogeneous epoxidation catalyst and removing during t_{3.2} the gaseous stream from the epoxidation zone, wherein the pressure in the epoxidation zone is in the range of from 0.01 to 0.10 MPa, preferably in the range of from 0.02 to 0.08 MPa, more preferably in the range of from 0.03 to 0.07 MPa.

Preferably, t_{3.1} is a time span in the range of 15 to 25% of t₃ and t_{3.2} is a time span in the range of from 75 to 85% of t₃. In the context of the present invention, the period of time t₂ follows after t₁ and t₃ follows after t₂. Preferably, t₂ follows after t₁, t_{3.1} follows after t₂ and t_{3.2} follows after t_{3.1}.

According to a preferred embodiment of the shutdown method, the epoxidation reaction conditions comprise trickle bed conditions. According to another preferred embodiment of the shut-down method, the epoxidation reaction conditions comprise fixed bed conditions.

Conducting the shutdown method with steps (a) to (f) and preferably also (g), allows to maintain the oxygen (O₂) content in the effluent stream removed from the epoxidation zone during t₁ according to (b) and/or the effluent stream removed from the epoxidation zone during t₂ according to (d) and/or the gaseous stream removed from the epoxidation zone during t₃ according to (g), preferably in the effluent stream removed from the epoxidation zone during t₁ according to (b) and in the effluent stream removed from the epoxidation zone during t₂ according to (d) and in the gaseous stream removed from the epoxidation zone during t₃ according to (g) below a certain level. Preferably, any of these effluent streams as well as the gaseous stream has an O₂ content, which is at most 50%, preferably at most 25%, more preferably at most 10% higher than the O₂ content of an effluent stream obtained from epoxidation reaction during a normal-run stage, each based on the O₂ content of the respective effluent stream. More preferably, any of these effluent streams as well as the gaseous stream has an O₂ content, which is in the range of from 0 to 150%, more preferably in the range of from 0.1 to 125%, more preferably in the range of from 1 to 110% of the O₂ content of an effluent stream obtained from epoxidation reaction during a normal-run stage, each based on the O₂ content of the respective effluent stream.

### Separation Unit

According to a preferred embodiment of the shutdown method, the effluent stream removed from the epoxidation zone during t₁ according to (b) and/or the effluent stream removed from the epoxidation zone during t₂ according to (d) and/or the gaseous stream removed from the epoxidation zone during t₃ according to (g), each optionally after an intermediate step, is introduced into a separation unit at a pressure in the range of from 0.05 to 0.5 MPa, preferably in the range of from 0.08 to 0.15 MPa, more preferably in the range of from 0.09 to 0.12 MPa, thereby obtaining a gaseous stream (vent stream) comprising oxygen.

The gaseous stream obtained from the separation unit preferably has an oxygen content which is at most 50%, preferably at most 25%, more preferably at most 10% higher than the O₂ content of a gaseous stream obtained from the separation unit during a normal-run stage, each based on the total weight of the respective gaseous stream. More preferably, the gaseous stream has an O₂ content, which is in the range of from 0 to 150%, more preferably in the range of from 0.1 to 125%, more preferably in the range of from 1 to 110% of the O₂ content of an effluent stream obtained from epoxidation reaction during a normal-run stage, each based on the O₂ content of the respective gaseous stream.

According to a preferred embodiment of the shutdown method, in the range of from 90 to 100 weight-%, preferably in the range of from 95 to 100 weight-%, more preferably in the range of from 98 to 100 weight-%, of the gaseous stream obtained from the separation unit consist of oxygen, inert gas and optionally propylene, based on the total weight of the gaseous stream.

According to a preferred embodiment of the shutdown method, the separation unit is a separation column, which is preferably operated at a temperature at the top of the separation column in the range of from 34 to 40° C. and at a temperature at the bottom of the separation column in the range of from 70 to 80° C. According to a preferred embodiment of the shutdown method, the pressure is reduced in the intermediate step from the range of from 0.5 to 5.0 MPa, preferably in the range of from 1.5 to 3.0 MPa, more preferably in the range of from 1.8 to 2.8 MPa (epoxidation reaction conditions) to a pressure in the range of from 0.05 to 0.5 MPa, preferably in the range of from 0.08 to 0.15 MPa, more preferably in the range of from 0.09 to 0.12 MPa (separation conditions in separation unit).

### Additives

According to a preferred embodiment of the shutdown method, in (a) an additive is provided to the epoxidation zone, preferably an aqueous solution of an additive, so that the mixture formed in (a) comprises an additive.

According to a preferred embodiment of the shutdown method, providing the additive in (a) is stopped before expiry of t₁, preferably after a period of time which is in the range of from 0.1×t₁ to 0.8×t₁, more preferably after a period of time which is in the range of from 0.5×t₁ to 0.7×t₁, wherein more preferably, essentially no additive is added during (d) and/or during (g), more preferably no additive is added during (d) and during (g).

According to a preferred embodiment of the shutdown method, in the normal run stage, an additive is provided to the epoxidation zone, preferably an aqueous solution of an additive, so that the reaction mixture comprising olefin, hydrogen peroxide and organic solvent, which is subjected to epoxidation reaction conditions in the epoxidation zone comprises an additive.

According to a preferred embodiment of the shutdown method, the additive is selected from the group consisting of a potassium salt, ammonia, an ammonium salt, etidronic acid, a salt of etidronic acid and mixtures of two or more thereof, preferably selected from the group consisting of potassium dihydrogen phosphate, dipotassium hydrogen phosphate, potassium formate, potassium acetate, potassium hydrogen carbonate, dipotassium etidronate, ammonium dihydrogen phosphate, diammonium hydrogen phosphate, ammonia and mixtures of two or more thereof, preferably form the group consisting pf potassium dihydrogen phosphate, dipotassium hydrogen phosphate, dipotassium etidronate, ammonia and mixtures of two or more thereof, wherein the additive more preferably comprises at least dipotassium hydrogen phosphate.

### Heterogeneous Epoxidation Catalyst

According to a preferred embodiment of the shutdown method, the heterogeneous epoxidation catalyst comprises a zeolitic material having a framework structure comprising Si, O and Ti.

According to a preferred embodiment of the shutdown method, the zeolitic material comprises Ti in an amount in the range of from 0.2 to 5 weight-%, preferably in the range of from 0.5 to 4 weight-%, more preferably in the range of from 1.0 to 3 weight-%, more preferably in the range of from 1.2 to 2.5 weight-%, more preferably in the range of from 1.4 to 2.2 weight-%, calculated as elemental Ti and based on the total weight of the zeolitic material. According to a preferred embodiment of the shutdown method, the zeolitic material having a framework structure comprising Si, O and Ti comprised in the epoxidation catalyst is a titanium zeolite having ABW, ACO, AEI, AEL, AEN, AET, AFG, AFI, AFN, AFO, AFR, AFS, AFT, AFX, AFY, AHT, ANA, APC, APD, AST, ASV, ATN, ATO, ATS, ATT, ATV, AWO, AWW, BCT, BEA, BEC, BIK, BOG, BPH, BRE, CAN, CAS, CDO, CFI, CGF, CGS, CHA, CHI, CLO, CON, CZP, DAC, DDR, DFO, DFT, DOH, DON, EAB, EDI, EMT, EPI, ERI, ESV, ETR, EUO, FAU, FER, FRA, GIS, GIU, GME, GON, GOO, HEU, IFR, ISV, ITE, ITH, ITQ, ITW, IWR, IWW, JBW, KFI, LAU, LEV, LIO, LOS, LOV, LTA, LTL, LTN, MAR, MAZ, MCM-22(S), MCM-36, MCM-56, MEI, MEL, MEP, MER, MIT-1, MMFI, MFS, MON, MOR, MSE, MSO, MTF, MTN, MTT, MTW, MWW, NAB, NAT, NEES, NON, NPO, OBW, OFF, OSI, OSO, PAR, PAU, PHI, PON, RHO, RON, RRO, RSN, RTE, RTH, RUT, RWR, RWY, SAO, SAS, SAT, SAV, SBE, SBS, SBT, SFE, SFF, SFG, SFH, SFN SFO, SGT, SOD, SSY, STF, STI, STT, TER, THO, TON, TSC, UEI, UFI, UOZ, USI, UTL, VET, VFI, VNI, VSV, WEI, WEN, YUG, ZON SVR, SVY framework structure or a mixed structure of two or more of these framework types; more preferably the zeolitic material having a framework structure comprising Si, O and Ti is a titanium zeolite having an MFI framework type, an MEL framework type, an MWW framework type, an MCM-22(S) framework type, an MCM-56 framework type, an IEZ-MWW framework type, an MCM-36 framework type, an ITQ framework type, a BEA framework type, a MOR framework type, or a mixed structure of two or more of these framework types; more preferably the zeolitic material having a framework structure comprising Si, O and Ti is a titanium zeolite having an MFI framework type or an MWW framework type; more preferably the zeolitic material having a framework structure comprising Si, O and Ti has framework type MFI; more preferably the zeolitic material having a framework structure comprising Si, O and Ti is a titanium silicalite-1 (TS-1).

According to a preferred embodiment of the shutdown method, the heterogeneous epoxidation catalyst further comprises a binder. According to a preferred embodiment of the shutdown method, the heterogeneous epoxidation catalyst is in the form of a molding, preferably in the form of an extrudate or a granule. According to a preferred embodiment of the shutdown method, from 95 to 100 weight-%, preferably from 98 to 100 weight-%, more preferably from 99 to 100 weight-%, more preferably from 99.5 to 100 weight-%, more preferably from 99.9 to 100 weight-% of the molding consist of the zeolitic material and the binder. According to a preferred embodiment of the shutdown method, from 95 to 100 weight-%, preferably from 98 to 100 weight-%, more preferably from 99 to 100 weight-%, more preferably from 99.5 to 100 weight-%, more preferably from 99.9 to 100 weight-% of the binder comprised in the molding consist of Si and O.

According to a preferred embodiment of the shutdown method, the heterogeneous epoxidation catalyst, preferably the molding, comprises the binder, calculated as SiO₂, in an amount in the range of from 2 to 90 weight-%, preferably in the range of from 5 to 70 weight-%, more preferably in the range of from 10 to 50 weight-%, more preferably in the range of from 15 to 30 weight-%, more preferably in the range of from 20 to 25 weight-%, based on the total weight of the epoxidation catalyst, preferably based on the total weight of the molding and/or wherein the heterogeneous epoxidation catalyst, preferably the molding, comprises the zeolitic material in an amount in the range of from 10 to 98 weight-%, preferably in the range of from 30 to 95 weight-more preferably in the in the range of from 50 to 90 weight-%, more preferably in the range of from 70 to 85 weight-%, more preferably in the range of from 75 to 80 weight-%, based on the total weight of the heterogeneous epoxidation catalyst, preferably based on the total weight of the molding.

### Hydrogen Peroxide

According to a preferred embodiment of the shutdown method, the hydrogen peroxide is provided as aqueous hydrogen peroxide solution, which preferably has a total organic carbon content (TOC) in the range of from 100 to 800 mg per kg hydrogen peroxide comprised in the aqueous hydrogen peroxide solution, preferably in the range of from 120 to 750 mg per kg hydrogen peroxide comprised in the aqueous hydrogen peroxide solution, more preferably in the range of from 150 to 700 mg per kg hydrogen peroxide comprised in the aqueous hydrogen peroxide solution, determined according to DIN EN 1484 (April 2019). According to a preferred embodiment of the shutdown method, the hydrogen peroxide has a pH in the range of from 0 to 3.0, preferably in the range of from 0.1 to 2.5, more preferably in the range of from 0.5 to 2.3, determined with a pH sensitive glass electrode according to CEFIC PEROXYGENS H₂O₂ AM-7160 standard (2003). According to a preferred embodiment of the shutdown method, the hydrogen peroxide comprises from 20 to 85 weight-%, preferably from 30 to 75 weight-%, more preferably from 40 to 70 weight-% of hydrogen peroxide, relative to the total weight of the aqueous hydrogen peroxide solution.

According to a preferred embodiment of the shutdown method, the hydrogen peroxide is ob-tained or obtainable from an anthraquinone process.

According to the inventive shutdown process, the first step of said shutdown method comprises, compared to a normal-run stage, that the feed of hydrogen peroxide to the reactor is stopped, so that in step (a) of the shutdown method olefin and organic solvent are provided for a period of time t₁ to the epoxidation zone, so that a mixture is formed, which is essentially free of hydrogen peroxide. Since the hydrogen peroxide is preferably provided as aqueous solution, stopping the hydrogen peroxide feed also means that no water is provided in step (a), i.e. the mixture formed in (a) is essentially free of water. “essentially free of water” means that the mixture formed in (a) comprises less than 1 weight-%, preferably less than 0.5 weight-%, more preferably less than 0.1 weight-%, of water based on the total weight of the mixture.

### Organic Solvent

According to a preferred embodiment of the shutdown method, the organic solvent is an organic epoxidation solvent, preferably the organic solvent is selected from the group consisting of alcohol, acetonitrile, propionitrile and mixtures of two or more thereof; more preferably selected from the group consisting of alcohol, acetonitrile and mixtures of alcohol and acetonitrile; more preferably the organic solvent comprises at least an alcohol, wherein the alcohol is preferably a C1 to C5 mono alcohol or a mixture of two or more C1 to C5 alcohols, more preferably the alcohol comprises at least methanol.

### Olefin

According to a preferred embodiment of the shutdown method, the olefin is a C2-C10 alkene, preferably a C2-C5 alkene, more preferably a C2-C4 alkene, more preferably ethylene (C2 alkene) or propylene (C3 alkene), more preferably propylene (C3 alkene).

### 2nd Aspect—Combined Process

In a second aspect the present invention relates to a process for preparing an olefin oxide comprising a normal run stage and a shutdown stage, wherein the normal run stage comprises
(A) providing olefin, hydrogen peroxide, water and organic solvent into an epoxidation zone comprising an heterogeneous epoxidation catalyst, so that a reaction mixture comprising olefin, hydrogen peroxide, water and organic solvent is formed;
(B) subjecting the reaction mixture from (A) to epoxidation reaction conditions in the epoxidation zone, thereby obtaining a mixture comprising olefin oxide, water and organic solvent;
(C) removing an effluent stream from the epoxidation zone, comprising olefin oxide, water and organic solvent;

wherein the shutdown stage comprises
(a) providing olefin and organic solvent for a period of time t₁ to the epoxidation zone, so that a mixture is formed, which is essentially free of hydrogen peroxide;
(b) contacting the mixture from (a) over t₁ under epoxidation reaction conditions in the epoxidation zone with the heterogeneous epoxidation catalyst;
(c) removing during t₁ an effluent stream from the epoxidation zone, the effluent stream comprising olefin, optionally olefin oxide and organic solvent.

According to a preferred embodiment of the process for preparing an olefin oxide, the epoxidation in (B) is carried out at an absolute pressure in the reaction zones in the range of from 0.5 to 5.0 MPa, preferably in the range of from 1.5 to 3.0 MPa, more preferably in the range of from 1.8 to 2.8 MPa. According to a preferred embodiment of the process for preparing an olefin oxide, the epoxidation in (B) is carried out at a temperature in the reaction zones in the range of from 20 to 75° C., preferably in the range of from 25 to 75° C., more preferably in the range of from 28 to 70° C., more preferably in the range of from 30 to 65° C.

According to a preferred embodiment of the process for preparing an olefin oxide, the weight ratio of olefin:hydrogen peroxide (w/w) in the reaction mixture formed in (A) is in the range of from 1:1 to 5:1, preferably in the range of from 1:1 to 2:1 or in the range of from 3:1 to 5:1. According to a preferred embodiment of the process for preparing an olefin oxide, the weight ratio of organic solvent:hydrogen peroxide (w/w) in the reaction mixture formed in (A) is in the range of from 15:1 to 5:1, preferably in the range of from 12:1 to 6:1, more preferably in the range of from 12:1 to 8.5:1 or in the range of from 8:1 to 6:1. According to a preferred embodiment of the process for preparing an olefin oxide, the weight ratio of organic solvent:olefin (w/w) in the reaction mixture formed in (A) is in the range of from 10:1 to 1:0.1, preferably in the range of from 9:1 to 1:1, more preferably in the range of from 7:1 to 4:1 or in the range of from 1.5:1 to 1:1.

In some preferred embodiments, the molar amount of olefin provided in (a) of the shut-down stage is in the range of 0.25×[(molar amount of olefin provided in (A)) minus (molar amount of hydrogen peroxide provided in (A))] to 3.0×[(molar amount of olefin provided in (A)) minus (molar amount of hydrogen peroxide provided in (A))], more preferably in the range of 0.5×[(molar amount of olefin provided in (A)) minus (molar amount of hydrogen peroxide provided in (A))] to 2.5×[(molar amount of olefin provided in (A)) minus (molar amount of hydrogen peroxide provided in (A))], more preferably in the range of 1.8×[molar amount of olefin provided in (A)) minus (molar amount of hydrogen peroxide provided in (A))] to 2.0×[(molar amount of olefin provided in (A)) minus (molar amount of hydrogen peroxide provided in (A))]. As described above in the section related to the shut-down stage, the weight ratio of organic solvent:olefin (w/w) in the mixture from (a) in some preferred embodiments of said shut-down stage is in the range of from 18:1 to 1:0.1, preferably in the range of from 15:1 to 1:1, more preferably in the range of from 12:1 to 7:1.

Details regarding olefin, hydrogen peroxide, organic solvent and heterogeneous epoxidation catalyst for (A), (B) and (C) of the normal-run stage are as disclosed above in the first section related to the shutdown method. No restrictions exist regarding the water used for the reaction mixture. It is conceivable to use, for example, water which is treated with NH₃ but water not having been treated with NH₃ can also be used. Preferably deionized water is used for the reaction mixture. The deionized water can be obtained using ion-exchangers of using condensate. Typical grades of deionized water are defined in ISO 3696 of 1987 and all grades described there can be used within the scope of this invention. The water may additionally contain traces of corrosion inhibiting additives like ammonia, hydrazine or hydroxylamine in which case it should have a pH value in the range of 7 to 9 (determined with a pH sensitive glass electrode according to CEFIC PEROXYGENS H₂O₂ AM-7160 standard (2003)). Preferably, the water used does not contain corrosion inhibiting additives. As indicated above in the 1st section related to the shutdown stage, the hydrogen peroxide is preferably provided as aqueous solution, i.e. the water is preferably provided in (A) as part of the aqueous hydrogen peroxide solution.

The olefin oxide comprised in the effluent stream, which is removed from the epoxidation zone according to (C), is preferably a C2-C10 alkylene oxide, more preferably a C2-C5 alkylene oxide, more preferably a C2-C4 alkylene oxide, more preferably a C2 or C3 alkylene oxide, more preferably propylene oxide (C3 alkylene oxide).

The term “reaction mixture” as used in this context of the present invention relates to a mixture containing the complete amount of olefin, the complete amount of hydrogen peroxide, the complete amount of organic solvent and optionally the complete amount of additive.

The optional additive is preferably comprised in the reaction mixture in the normal-run stage. During the shut-down stage, preferably the organic solvent provided in (d) is essentially free of additive, which means that the organic solvent provided in (d) comprises less than 1 weight-%, preferably less than 0.1 weight-%, more preferably less than 0.001 weight-%, of additive, based on the total weight of the organic solvent provided in (d) respectively. It is also preferred that during (g) of the shut-down method, no additive is introduced into the reactor. As disclosed above in the section related to the shutdown stage, at least for a part of the period of time t₁, the mixture provided in (a) is also essentially free of additive.

Preferably, at least three individual feed streams are used in the normal-run stage, at least one of which is the feed with which the organic solvent is introduced into the reactor, at least one of which is the feed with which the olefin is introduced into the reactor and at least one of which is the feed with which the hydrogen peroxide and the water are introduced into the reactor. Op-tionally, there is at least one further individual stream, which is the feed with which the additive is introduced.

The individual feed streams can be combined and introduced into the reactor as one single feed stream or as combined feed streams such as, for example, a feed stream containing organic solvent hydrogen peroxide and water and a feed stream containing olefin, or a feed stream con-taining organic solvent and olefin and a feed stream containing hydrogen peroxide and water, or a feed stream containing organic solvent and a feed stream containing olefin and a feed stream containing hydrogen peroxide and water. The optional additive can be added as separate feed stream or can be mixed to one or more of the above-described feed streams. If more than one feed stream is employed, the individual feed streams are either mixed before they are introduced in the reactor or suitably mixed after having been introduced into the reactor.

According to a preferred embodiment of the process for preparing an olefin oxide, organic solvent, hydrogen peroxide, water olefin and optional additive are mixed before entering the reactor, so that a single feed stream comprising organic solvent, hydrogen peroxide, water olefin and optional additive is introduced into the reactor, which comprises the epoxidation zone. Preferably, the respective individual streams are suitably mixed to obtain a reaction feed, which con-sists of at least one liquid phase. Even more preferably, the individual streams are suitably mixed to obtain a feed stream, which consists of one liquid phase.

According to a preferred embodiment of the process for preparing an olefin oxide, the epoxidation reaction conditions according to (B) comprise fixed bed conditions.

According to a preferred embodiment of the process for preparing an olefin oxide, the epoxidation reaction conditions according to (B) comprise trickle bed conditions. According to this embodiment, organic solvent, water and hydrogen peroxide and optionally additive, are mixed before entering the reactor. The olefin is introduced into the reactor as separate stream. The reaction mixture is then formed in the reactor.

According to a preferred embodiment of the process for preparing an olefin oxide, the shutdown stage further comprises
(d) providing organic solvent for a period of time t₂ to the epoxidation zone, wherein the organic solvent is essentially free of hydrogen peroxide and of olefin;
(e) contacting the organic solvent from (d) over t₂ under epoxidation reaction conditions in the epoxidation zone with the heterogeneous epoxidation catalyst,
(f) removing during t₂ an effluent stream from the epoxidation zone, the effluent stream comprising organic solvent.

According to a preferred embodiment of the process for preparing an olefin oxide, the shutdown stage further comprises
(g) providing a gaseous stream and passing said gaseous stream for a period of time t₃ into the epoxidation zone comprising the heterogeneous epoxidation catalyst and removing during t₃ the gaseous stream from the epoxidation zone.

According to a preferred embodiment of the process for preparing an olefin oxide, (a) comprises
(a.1) providing olefin and organic solvent,
(a.2) combining olefin and organic solvent so that a mixture comprising olefin and organic solvent is formed, which is essentially free of hydrogen peroxide;
(a.3) feeding the mixture obtained in (a.2) for a period of time t₁ to the epoxidation zone.

According to a preferred embodiment of the process for preparing an olefin oxide, step (g) comprises
(g.1) providing a gaseous stream and passing said gaseous stream for a period of time t_{3.1} into the epoxidation zone comprising the heterogeneous epoxidation catalyst and removing during t_{3.1} the gaseous stream from the epoxidation zone, wherein the pressure in the epoxidation zone is in the range of from 0.5 to 5.0 MPa, preferably in the range of from 1.5 to 3.0 MPa, more preferably in the range of from 1.8 to 2.8 MPa;
(g.2) providing a gaseous stream and passing said gaseous stream for a period of time t_{3.2} into the epoxidation zone comprising the heterogeneous epoxidation catalyst and removing during t_{3.2} the gaseous stream from the epoxidation zone, wherein the pressure in the epoxidation zone is in the range of from 0.01 to 0.10 MPa, preferably in the range of from 0.02 to 0.08 MPa, more preferably in the range of from 0.03 to 0.07 MPa.

Details regarding the chemicals used such as olefin, hydrogen peroxide, organic solvent and heterogeneous epoxidation catalyst, as well as the optional additive and the separation unit for (a) to (g) in the context of the process for preparing an olefin oxide of the second aspect are as disclosed above in the first section related to the shutdown method. Also for the conditions of the shutdown stage of the process for preparing an olefin oxide, such as epoxidation conditions, flow rate and composition of gaseous stream, time spans (t₁, t₂, t₃, t_{3.1}, t_{3.2}), details apply as disclosed above in the first section related to the shutdown method. The same also applies for definitions given in the first section, such as the definitions of “Essentially free of hydrogen peroxide” and “Essentially free of hydrogen peroxide and of olefin”.

Also in the context of the second aspect, it applies that preferably at least one of (a), (b) and (c) preferably (a), (b) and (c), are carried out continuously and/or wherein at least one of (d), (e) and (f) preferably (d), (e) and (f) are carried out continuously and/or wherein (g) is carried out continuously; wherein more preferably at least (a), (b), (c), (d), (e) and (f) are carried out continuously, wherein more preferably (a), (b), (c), (d), (e), (f) and (g) are carried out continuously.

Also in the context of the second aspect, it applies that the normal run stage is preferably carried out in continuous mode.

The present invention is further illustrated by the following set of embodiments and combinations of embodiments resulting from the dependencies and back-references as indicated. In particular, it is noted that in each instance where a range of embodiments is mentioned, for example in the context of a term such as “any one of embodiments (1) to (4)”, every embodiment in this range is meant to be explicitly disclosed for the skilled person, i.e. the wording of this term is to be understood by the skilled person as being synonymous to “any one of embodiments (1), (2), (3) and (4)”. Further, it is explicitly noted that the following set of embodiments is not the set of claims determining the extent of protection, but represents a suitably structured part of the description directed to general and preferred aspects of the present invention.
1. A shutdown method for a process for preparing an olefin oxide comprising a normal run stage, wherein the normal run stage comprises providing olefin, hydrogen peroxide and organic solvent into an epoxidation zone comprising an heterogeneous epoxidation catalyst, so that a reaction mixture comprising olefin, hydrogen peroxide and organic solvent is formed and subjecting the reaction mixture to epoxidation reaction conditions in the epoxidation zone, thereby obtaining a mixture comprising olefin oxide and organic solvent; wherein the shutdown method comprises
   (g) providing olefin and organic solvent for a period of time t₁ to the epoxidation zone, so that a mixture is formed, which is essentially free of hydrogen peroxide;
   (h) contacting the mixture from (a) over t₁ under epoxidation reaction conditions in the epoxidation zone with the heterogeneous epoxidation catalyst;
   (i) removing during t₁ an effluent stream from the epoxidation zone, the effluent stream comprising olefin and organic solvent.
2. The shutdown method of embodiment 1, wherein the shutdown stage further comprises
   (j) providing organic solvent for a period of time t₂ to the epoxidation zone, wherein the organic solvent is essentially free of hydrogen peroxide and of olefin;
   (k) contacting the organic solvent from (d) over t₂ under epoxidation reaction conditions in the epoxidation zone with the heterogeneous epoxidation catalyst,
   (l) removing during t₂ an effluent stream from the epoxidation zone, the effluent stream comprising organic solvent.
3. The shutdown method of embodiment 1 or 2, wherein the shutdown stage further comprises
   (g) providing a gaseous stream and passing said gaseous stream for a period of time t₃ into the epoxidation zone comprising the heterogeneous epoxidation catalyst and removing during t₃ the gaseous stream from the epoxidation zone.
4. The shutdown method of any one of embodiments 1 to 3, wherein the gaseous stream used in (g) comprises at least 90 volume-%, more preferably at least 95 volume-%, more preferably at least 98 volume-% of nitrogen, based on the total volume of the gaseous stream.
5. The shutdown method of any one of embodiments 1 to 4, wherein at least one of (a), (b) and (c) preferably (a), (b) and (c), are carried out continuously and/or wherein at least one of (d), (e) and (f) preferably (d), (e) and (f) are carried out continuously and/or wherein (g) is carried out continuously; wherein more preferably at least (a), (b), (c), (d), (e) and (f) are carried out continuously, wherein more preferably (a), (b), (c), (d), (e), (f) and (g) are carried out continuously.
6. The shutdown method of any one of embodiments 1 to 5, wherein the normal run stage is carried out in continuous mode.
7. The shutdown method of any one of embodiments 1 to 5, wherein (a) of the shutdown stage comprises
   (a.1) providing olefin and organic solvent,
   (a.2) combining olefin and organic solvent so that a mixture comprising olefin and organic solvent is formed, which is essentially free of hydrogen peroxide;
   (a.3) feeding the mixture obtained in (a.2) for a period of time t₁ to the epoxidation zone.
8. The shutdown method of any one of embodiments 1 to 7, wherein t₁ is a period of time in the range of from 1 minute to 10 hours, preferably in the range of from 10 minutes to 5 hours, more preferably in the range of from 30 minutes to 2 hours.
9. The shutdown method of any one of embodiments 2 to 8, wherein t₂ is a period of time in the range of from 1 minute to 10 hours, preferably in the range of from 10 minutes to 5 hours, more preferably in the range of from 30 minutes to 2 hours.
10. The shutdown method of any one of embodiments 3 to 9, wherein t₃ is a period of time in the range of from 1 to 48 hours, preferably in the range of from 10 to 36 hours, more preferably in the range of from 20 to 30 hours.
11. The shutdown method of any one of embodiments 1 to 10, wherein the contacting under epoxidation reaction conditions in the epoxidation zone with the heterogeneous epoxidation catalyst in (b) and/or (f) is carried out at an absolute pressure in the epoxidation zone in the range of from 0.5 to 5.0 MPa, preferably in the range of from 1.5 to 3.0 MPa, more preferably in the range of from 1.8 to 2.8 MPa.
12. The shutdown method of any one of embodiments 1 to 11, wherein the contacting under epoxidation reaction conditions in the epoxidation zone with the heterogeneous epoxidation catalyst in (b) and/or (f) is carried out at a temperature in the epoxidation zone in the range of from 20 to 75° C., preferably in the range of from 22 to 75° C., more preferably in the range of from 24 to 70° C., more preferably in the range of from 25 to 65° C.
13. The shutdown method of any one of embodiments 1 to 12, wherein the epoxidation reaction conditions comprise trickle bed conditions.
14. The shutdown method of any one of embodiments 1 to 12, wherein the epoxidation reaction conditions comprise fixed bed conditions.
15. The shutdown method of any one of embodiments 1 to 14, wherein the effluent stream removed from the epoxidation zone during t₁ according to (b) and/or the effluent stream removed from the epoxidation zone during t₂ according to (d) and/or the gaseous stream removed from the epoxidation zone during t₃ according to (g), each optionally after an intermediate step, is introduced into a separation unit at a pressure in the range of from 0.05 to 0.5 MPa, preferably in the range of from 0.08 to 0.15 MPa, more preferably in the range of from 0.09 to 0.12 MPa, thereby obtaining a gaseous stream (vent stream) comprising oxygen.
16. The shutdown method of embodiment 15, wherein in the range of from 90 to 100 weight-%, preferably in the range of from 95 to 100 weight-%, more preferably in the range of from 98 to 100 weight-%, of the gaseous stream obtained from the separation unit consist of oxygen, inert gas and optionally propylene, based on the total weight of the gaseous stream.
17. The shutdown method of embodiment 15 or 16, wherein the separation unit is a separation column, which is preferably operated at a temperature at the top of the separation column in the range of from 34 to 40° C. and at a temperature at the bottom of the separation column in the range of from 70 to 80° C.
18. The shutdown method of any one of embodiments 15 to 17, wherein in the intermediate step, the pressure is reduced from the range of from 0.5 to 5.0 MPa, preferably in the range of from 1.5 to 3.0 MPa, more preferably in the range of from 1.8 to 2.8 MPa (epoxidation reaction conditions) to a pressure in the range of from 0.05 to 0.5 MPa, preferably in the range of from 0.08 to 0.15 MPa, more preferably in the range of from 0.09 to 0.12 MPa (separation conditions in separation unit).
19. The shutdown method of any one of embodiments 1 to 18, wherein in (a) an additive is provided to the epoxidation zone, preferably an aqueous solution of an additive, so that the mixture formed in (a) comprises an additive.
20. The shutdown method of embodiment 19, wherein providing the additive in (a) is stopped before expiry of t₁, preferably after a period of time which is in the range of from 0.1×t₁ to 0.8×t₁, more preferably after a period of time which is in the range of from 0.5×t₁ to 0.7×t₁, wherein more preferably, essentially no additive is added during (d) and/or during (g), more preferably no additive is added during (d) and during (g).
21. The shutdown method of any one of embodiments 1 to 20, wherein in the normal run stage, an additive is provided to the epoxidation zone, preferably an aqueous solution of an additive, so that the reaction mixture comprising olefin, hydrogen peroxide and organic solvent, which is subjected to epoxidation reaction conditions in the epoxidation zone comprises an additive.
22. The shutdown method of any one pf embodiments 19 to 21, wherein the additive is selected from the group consisting of potassium salt, ammonia, ammonium salt, etidronic acid, salt of etidronic acid and mixtures of two or more thereof, preferably selected from the group consisting of potassium dihydrogen phosphate, dipotassium hydrogen phosphate, potassium formate, potassium acetate, potassium hydrogen carbonate, dipotassium etidronate, ammonium dihydrogen phosphate, diammonium hydrogen phosphate, ammonia and mixtures of two or more thereof, more preferably form the group consisting pf potassium dihydrogen phosphate, dipotassium hydrogen phosphate, dipotassium etidronate, ammonia and mixtures of two or more thereof, wherein the additive more preferably comprises at least dipotassium hydrogen phosphate.
23. The shutdown method of any one of embodiments 1 to 22, wherein the heterogeneous epoxidation catalyst comprises a zeolitic material having a framework structure comprising Si, O and Ti.
24. The shutdown method of embodiment 23, wherein the zeolitic material comprises Ti in an amount in the range of from 0.2 to 5 weight-%, preferably in the range of from 0.5 to 4 weight-%, more preferably in the range of from 1.0 to 3 weight-%, more preferably in the range of from 1.2 to 2.5 weight-%, more preferably in the range of from 1.4 to 2.2 weight-%, calculated as elemental Ti and based on the total weight of the zeolitic material.
25. The shutdown method of embodiment 23 or 24, wherein the zeolitic material having a framework structure comprising Si, O and Ti comprised in the epoxidation catalyst is a titanium zeolite having ABW, ACO, AEI, AEL, AEN, AET, AFG, AFI, AFN, AFO, AFR, AFS, AFT, AFX, AFY, AHT, ANA, APC, APD, AST, ASV, ATN, ATO, ATS, ATT, ATV, AWO, AWW, BCT, BEA, BEC, BIK, BOG, BPH, BRE, CAN, CAS, CDO, CFI, CGF, CGS, CHA, CHI, CLO, CON, CZP, DAC, DDR, DFO, DFT, DOH, DON, EAB, EDI, EMT, EPI, ERI, ESV, ETR, EUO, FAU, FER, FRA, GIS, GIU, GME, GON, GOO, HEU, IFR, ISV, ITE, ITH, ITQ, ITW, IWR, IWW, JBW, KFI, LAU, LEV, LIO, LOS, LOV, LTA, LTL, LTN, MAR, MAZ, MCM-22(S), MCM-36, MCM-56, MEI, MEL, MEP, MER, MIT-1, MMFI, MFS, MON, MOR, MSE, MSO, MTF, MTN, MTT, MTW, MWW, NAB, NAT, NEES, NON, NPO, OBW, OFF, OSI, OSO, PAR, PAU, PHI, PON, RHO, RON, RRO, RSN, RTE, RTH, RUT, RWR, RWY, SAO, SAS, SAT, SAV, SBE, SBS, SBT, SFE, SFF, SFG, SFH, SFN SFO, SGT, SOD, SSY, STF, STI, STT, TER, THO, TON, TSC, UEI, UFI, UOZ, USI, UTL, VET, VFI, VNI, VSV, WEI, WEN, YUG, ZON SVR, SVY framework structure or a mixed structure of two or more of these framework types; more preferably the zeolitic material having a framework structure comprising Si, O and Ti is a titanium zeolite having an MFI framework type, an MEL framework type, an MWW framework type, an MCM-22(S) framework type, an MCM-56 framework type, an IEZ-MWW framework type, an MCM-36 framework type, an ITQ framework type, a BEA framework type, a MOR framework type, or a mixed structure of two or more of these framework types; more preferably the zeolitic material having a framework structure comprising Si, O and Ti is a titanium zeolite having an MFI framework type, or an MWW framework type; more preferably the zeolitic material having a framework structure comprising Si, O and Ti has framework type MFI; more preferably the zeolitic material having a framework structure comprising Si, O and Ti is a titanium silicalite-1 (TS-1).
26. The shutdown method of any one of embodiments 23 to 25, wherein the heterogeneous epoxidation catalyst further comprises a binder.
27. The shutdown method of embodiment 26, wherein the heterogeneous epoxidation catalyst is in the form of a molding, preferably in the form of an extrudate or a granule.
28. The shutdown method of embodiment 26 or 27, wherein from 95 to 100 weight-%, preferably from 98 to 100 weight-%, more preferably from 99 to 100 weight-%, more preferably from 99.5 to 100 weight-%, more preferably from 99.9 to 100 weight-% of the molding consist of the zeolitic material and the binder.
29. The shutdown method of any one of embodiments 26 to 28, wherein from 95 to 100 weight-%, preferably from 98 to 100 weight-%, more preferably from 99 to 100 weight-%, more preferably from 99.5 to 100 weight-%, more preferably from 99.9 to 100 weight-% of the binder comprised in the molding consist of Si and O.
30. The shutdown method of embodiment 29, wherein the heterogeneous epoxidation catalyst, preferably the molding, comprises the binder, calculated as SiO₂, in an amount in the range of from 2 to 90 weight-%, preferably in the range of from 5 to 70 weight-%, more preferably in the range of from 10 to 50 weight-%, more preferably in the range of from 15 to 30 weight-%, more preferably in the range of from 20 to 25 weight-%, based on the total weight of the epoxidation catalyst, preferably based on the total weight of the molding and/or wherein the heterogeneous epoxidation catalyst, preferably the molding, comprises the zeolitic material in an amount in the range of from 10 to 98 weight-%, preferably in the range of from 30 to 95 weight-%, more preferably in the in the range of from 50 to 90 weight-%, more preferably in the range of from 70 to 85 weight-%, more preferably in the range of from 75 to 80 weight-%, based on the total weight of the heterogeneous epoxidation catalyst, preferably based on the total weight of the molding.
31. The shutdown method of any one of embodiments 1 to 30, wherein the hydrogen peroxide is provided as aqueous hydrogen peroxide solution, which preferably has a total organic carbon content (TOC) in the range of from 100 to 800 mg per kg hydrogen peroxide comprised in the aqueous hydrogen peroxide solution, preferably in the range of from 120 to 750 mg per kg hydrogen peroxide comprised in the aqueous hydrogen peroxide solution, more preferably in the range of from 150 to 700 mg per kg hydrogen peroxide comprised in the aqueous hydrogen peroxide solution, determined according to DIN EN 1484 (April 2019).
32. The shutdown method of any one of embodiments 1 to 31, wherein the hydrogen peroxide has a pH in the range of from 0 to 3.0, preferably in the range of from 0.1 to 2.5, more preferably in the range of from 0.5 to 2.3, determined with a pH sensitive glass electrode according to CEFIC PEROXYGENS H₂O₂ AM-7160 standard (2003).
33. The shutdown method of any one of embodiments 1 to 32, wherein the hydrogen peroxide comprises from 20 to 85 weight-%, preferably from 30 to 75 weight-%, more preferably from 40 to 70 weight-% of hydrogen peroxide, relative to the total weight of the aqueous hydrogen peroxide solution.
34. The shutdown method of any one of embodiments 1 to 33, wherein the hydrogen peroxide is obtained or obtainable from an anthraquinone process.
35. The shutdown method of any one of embodiments 1 to 34, wherein the organic solvent is an organic epoxidation solvent, preferably the organic solvent is selected from the group consisting of alcohol, acetonitrile, propionitrile and mixtures of two or more thereof; more preferably selected from the group consisting of alcohol, acetonitrile and mixtures of alcohol and acetonitrile; more preferably the organic solvent comprises at least an alcohol, wherein the alcohol is preferably a C1 to C5 mono alcohol or a mixture of two or more C1 to C5 alcohols, more preferably the alcohol comprises at least methanol.
36. The shutdown method of any one of embodiments 1 to 35, wherein the olefin is a C2-C10 alkene, preferably a C2-C5 alkene, more preferably a C2-C4 alkene, more preferably a ethylene or propylene, more preferably propylene.
37. A process for preparing an olefin oxide comprising a normal run stage and a shutdown stage, wherein the normal run stage comprises
   (A) providing olefin, hydrogen peroxide, water and organic solvent into an epoxidation zone comprising an heterogeneous epoxidation catalyst, so that a reaction mixture comprising olefin, hydrogen peroxide, water and organic solvent is formed;
   (B) subjecting the reaction mixture from (A) to epoxidation reaction conditions in the epoxidation zone, thereby obtaining a mixture comprising olefin oxide, water and organic solvent;
   (C) removing an effluent stream from the epoxidation zone, comprising olefin oxide, water and organic solvent;
   wherein the shutdown stage comprises
   (a) providing olefin and organic solvent for a period of time t₁ to the epoxidation zone, so that a mixture is formed, which is essentially free of hydrogen peroxide;
   (b) contacting the mixture from (a) over t₁ under epoxidation reaction conditions in the epoxidation zone with the heterogeneous epoxidation catalyst;
   (c) removing during t₁ an effluent stream from the epoxidation zone, the effluent stream comprising olefin, optionally olefin oxide and organic solvent.
38. The process for preparing an olefin oxide according to embodiment 37, wherein the epoxidation in (B) is carried out at an absolute pressure in the reaction zones in the range of from 0.5 to 5.0 MPa, preferably in the range of from 1.5 to 3.0 MPa, more preferably in the range of from 1.8 to 2.8 MPa.
39. The process for preparing an olefin oxide according to embodiment 37 or 38, wherein the epoxidation in (B) is carried out at a temperature in the reaction zones in the range of from 20 to 75° C., preferably in the range of from 25 to 75° C., more preferably in the range of from 28 to 70° C., more preferably in the range of from 30 to 65° C.
40. The process for preparing an olefin oxide according to any one of embodiments 37 to 39, wherein the weight ratio of olefin:hydrogen peroxide (w/w) in the reaction mixture formed in (A) is in the range of from 1:1 to 5:1, preferably in the range of from 1:1 to 2:1 or in the range of from 3:1 to 5:1.
41. The process for preparing an olefin oxide according to any one of embodiments 37 to 40, wherein the weight ratio of organic solvent:hydrogen peroxide (w/w) in the reaction mixture formed in (A) is in the range of from 15:1 to 5:1, preferably in the range of from 12:1 to 6:1, more preferably in the range of from 12:1 to 8.5:1 or in the range of from 8:1 to 6:1.
42. The process for preparing an olefin oxide according to any one of embodiments 37 to 41, wherein the weight ratio of organic solvent:olefin (w/w) in the reaction mixture formed in (A) is in the range of from 10:1 to 1:0.1, preferably in the range of from 9:1 to 1:1, more preferably in the range of from 7:1 to 4:1 or in the range of from 1.5:1 to 1:1.
43. The process for preparing an olefin oxide according to any one of embodiments 37 to 42, wherein the epoxidation reaction conditions according to (B) comprise fixed bed conditions.
44. The process for preparing an olefin oxide according to any one of embodiments 37 to 42, wherein the epoxidation reaction conditions according to (B) comprise trickle bed conditions.
45. The process for preparing an olefin oxide according to any one of embodiments 34 to 44, wherein the shutdown stage further comprises
   (d) providing organic solvent for a period of time t₂ to the epoxidation zone, wherein the organic solvent is essentially free of hydrogen peroxide and of olefin;
   (e) contacting the organic solvent from (d) over t₂ under epoxidation reaction conditions in the epoxidation zone with the heterogeneous epoxidation catalyst,
   (f) removing during t₂ an effluent stream from the epoxidation zone, the effluent stream comprising organic solvent.
46. The process for preparing an olefin oxide according to any one of embodiments 34 to 44, wherein the shutdown stage further comprises
   (g) providing a gaseous stream and passing said gaseous stream for a period of time t₃ into the epoxidation zone comprising the heterogeneous epoxidation catalyst and removing during t₃ the gaseous stream from the epoxidation zone.

The present invention is further illustrated by the following reference examples, comparative examples, and examples.

### EXAMPLES

Chemicals

| | |
|---|---|
| Methanol | CH₃OH |
| Propylene | C₃H₅ (C3) |
| Oxygen | O₂ |
| Nitrogen | N₂ |
| Hydrogen peroxide | H₂O₂ |
| Dipotassium hydrogen phosphate | K₂HPO₄ |
| tetra-n-propylammonium hydroxide | TPAOH |
| Tetraethoxysilane | TEOS |
| tetraethyl orthotitanate | TEOT |
| carboxymethyl cellulose (Mw = 15,000 g) | Walocel ® |
| Colloidal silica 40 weight-% in water | Ludox ® AS-40 |

### Reference Example 1: Determination of Oxygen and Nitrogen Concentration

The flow of the of the vent stream was measured by using a Brunkhorst flowmeter and the oxygen concentration was measured by using a Dräger Polytron 7000 detector with electrochemical sensor.

### Reference Example 2: Preparation of a Titanium Containing Zeolite (TS-1)

In a reaction vessel, 550 kg deionised water were provided and stirred. 400 kg TPAOH (tetra-n-propylammonium hydroxide) were added under stirring. Stirring was continued for 1 h. The resulting mixture was transferred in a suitable vessel. The reaction vessel was washed twice with 2000 l deionised water in total. In the washed reaction vessel, 300 kg TEOS (tetraethoxysilane) were provided and stirred. A mixture of 80 kg TEOS and 16 kg TEOT (tetraethyl orthotitanate) was added to the 300 kg TEOS. The remaining 340 kg TEOS were added.

Subsequently, the TPAOH solution was added and the resulting mixture was stirred for another hour. Then, the reaction vessel was heated and the ethanol obtained was separated by distillation. When the internal temperature of the vessel had reached 95° C., the reaction vessel was cooled. 1143 kg water were added to the resulting suspension in the vessel and the mixture was stirred for another hour. Crystallization was performed at 175° C. within 24 h at autogenous pressure. The obtained titanium silicalite-1 crystals were separated, dried and calcined at a temperature of 500° C. in air.

The obtained powder and Walocel® were mixed in a muller and mixed for 5 min. Within 10 min, the polystyrene dispersion was continuously added. Subsequently, 15 l Ludox® AS-40 were continuously added. The resulting mixture was mixed for 5 min and polyethylene oxide was continuously added within 15 min, followed by mixing for 10 min. Then, water was added. The formable mass was extruded through a matrix having circular holes with a diameter of 1.5 mm. The obtained strands were dried in a band drier at a temperature of 120° C. for 2 h and calcined at a temperature of 550° C. in lean air (100 m³/h air/100 m³/h nitrogen). The yield was 89 kg extrudates.

For the subsequent water treatment of the extrudates, 880 kg deionised water were filled in a respective stirred vessel and the extrudates were added. At a pressure of 84 mbar, the vessel was heated to an internal temperature of from 139 to 143° C. The resulting pressure was in the range of from 2.1 to 2.5 bar. Water treatment was carried out for 36 h. The extrudates were separated by filtration, dried for 16 h at 123° C. in air, heated to a temperature of 470° C. with 2° C./min and kept at a temperature of 490° C. in air for 5 h. The yield was 81.2 kg.

### Reference Example 3: Experimental Setup for Example 1 and Comparative Example 1

A TS-1 catalyst as obtained according to Reference Example 2 above was loaded into a reaction tube of a mini-plant with a length of 180 cm and a volume of 300 ml. The tube diameter was 0.75 inch (1.905 cm), with a wall thickness of 0.07 inch (0.19 cm). In the center of the reaction tube a smaller (0.125 inch (0.3175 cm)) tube was installed, containing thermoelements for measuring the temperature over the catalyst bed.

Feed-materials: 54 g/h Propylene (liquid)
94 g/h aqueous H₂O₂ solution (40 weight-% H₂O₂)

Solvent: 370 g/h Methanol

Additive-solution: 4 g/h aqueous K₂HPO₄ solution (0.3 weight-% K₂HPO₄)
(flow adjusted to maintain 130 micromole K⁺/(mole H₂O₂))

Propylene was stored in 50 l gas bottles, containing dip tubes, facilitating the transfer to the mini-plant by means of 25 bar nitrogen pressure. The precise amount was measured using a Brunkhorst flow meter with a 0-500 g/h range and the flow is controlled by means of a Flowserve control-valve. Hydrogen peroxide was transferred into the reactor using a Grundfos pump DME2. The amount was determined using a balance. The measurement showed liters/minute. The respective additive solution was fed to the reactor, using an hydrogen peroxide LC pump.

The precise amount was determined using a balance. For feeding the methanol a Lewa pump with a range of 0-1500 ml/h was used. Feed control was accomplished using a Lewa KMM. Nitrogen was fed using a Flowserve control-valve. The amount was measured using a Brunkhorst flow meter with a range of 0-200 NI/h. “NI/h” means norm liter per hour, wherein 1 norm liter is the amount of gas, which fills 1 liter at 0° C. and 1013 mbar (see DIN 1343 from January 1990).

Methanol, propylene, aqueous hydrogen peroxide solution and aqueous K₂HPO₄ additive solution entered the reactor tube via a static mixer [0.25 inch (0.635 cm)-mixer], so that a combined feed stream was formed, wherein the feed direction was from the bottom to the top direction of the reaction tube.

The experiments were carried out at an absolute pressure of 20 bar. The temperature in the reactor was controlled to ensure a H₂O₂ conversion of approximately 90% by using a cooling jacket circuit with oil. Typical start temperature was approximately 40-45° C. Then the temperature was slowly ramped up to approximately 60-65° C. over a run-time of 600 to 700 hours. At the beginning of the run the reactor was cooled as the exothermic heat would overheat the reactor otherwise. Towards the end of the run the reactor was heated to reach a temperature of 60-65° C.

The reactor effluent was passed through a 2 micrometer filter to remove fine (catalyst) particles before it was passed into the first separator. The bottom level valve controlled a level of 25% in the first separator, while the upper pressure valve set a pressure of 20 bars over the entire upstream reaction system. The second separator was also operated at a liquid level of 25%, while the upper pressure valve reduced the pressure to 2 bars. This lower pressure served for allowing the flashing of unconverted propylene, allowing a safe sample taking and having an additional safety buffer. The two separators had a volume of 2 liters each and were kept at a temperature of 5° C., using cooling water. A nitrogen stream of a specific gas flow was fed through the entire system (reactor→1^{st} separator→2^{nd} separator→vent-system) to maintain a sufficient gas flow in the direction of the vent to ascertain that traces of oxygen, formed by partial decomposition of H₂O₂ were flashed out and could be analyzed at the end of the vent pipe. A flowmeter was installed in the vent line, though which the gaseous vent stream coming from the 2nd separator flows, to measure the flow and quantify the amount of oxygen.

### Example 1: Shutdown Process with Stop of Feed of Aqueous Hydrogen Peroxide Solution First

An epoxidation of propylene was carried out according to Reference Example 3 for 600 hours, which represents a so called “normal run”. After 600 hours, an intermediate shutdown was initiated in that the feed of aqueous hydrogen peroxide to the static mixer was stopped. Shortly thereafter, also the additive solution feed (K₂HPO₄) was stopped. A combined feed stream comprising methanol and propylene and was further feed to the reactor for 60 minutes under a continuous N₂ gas flow of 5 NI/h. Any residual amounts of hydrogen peroxide were consumed in this way. The cooling jacket circuit was still run with the last temperature set point. After these 60 minutes, the feed of propylene to the static mixer was stopped, so that the combined feed stream entering the reactor after the static mixer essentially consisted of methanol, which was fed also under a continuous N₂ gas flow of 5 NI/h. The cooling jacket circuit was still run with the last temperature set point. After further 60 minutes, methanol stream was stopped. The hot oil was circulating at the last set point until 2 h and interrupted just before the plant depressurization and the start with nitrogen purging. The reactor was slowly depressurised to remove the residual propylene in the vent line and was flushed with nitrogen (N₂ purge, gas flow 100 NI/h) for two hours to remove methanol and dry the catalyst. Total time for the intermediate shutdown were four hours.

Afterwards, the reactor was restarted and epoxidation of propylene was conducted in accordance with Reference Example 3 for three hours. After three hours, the reactor was finally shutdown following the procedure as described above for the intermediate shutdown. Total time for the final shutdown were also four hours.

The O₂ concentration in the vent stream at the outlet of the second separator was measured according to Reference Example 1 over the complete time of epoxidation, intermediate shutdown, epoxidation and final shutdown (“O₂ concentration after the second separator”). The results are graphically depicted in FIG. 1, wherein the O₂ concentration indicated in volume-% based on the total volume of the vent stream on the y-axis is shown versus the time in hours on the x-axis of the intermediate shutdown and during the epoxidation phase between intermediate and final shutdown. “0 h” on the x-axis indicated the point in time after 600 hours of epoxidation and directly at the stop of the feed of aqueous hydrogen peroxide solution. The volume-% value indicated at 0 h (0.4 volume-%) represents the O₂ content in the vent stream over the complete time of epoxidation, i.e. during a normal run.

### Comparative Example 1: Shutdown Process with Stop of Propylene Feed First

An epoxidation of propylene was carried out according to Reference Example 3 for 600 hours, which represents a so called “normal run. After 600 hours, an intermediate shutdown was initiated in that the feed of propylene to the static mixer was stopped A feed stream comprising methanol and aqueous hydrogen peroxide and aqueous K₂HPO₄ stream was further fed to the reactor for 60 minutes under a continuous N₂ gas flow of 5 NI/h. The cooling jacket circuit was still run with the last temperature set point. After these 60 minutes, the feed of aqueous hydrogen peroxide to the static mixer was stopped, and shortly thereafter, also the aqueous K₂HPO₄ stream was stopped, so that the feed stream entering the reactor after the static mixer essentially consisted of methanol, which was fed also under a continuous N₂ gas flow of 5 NI/h. The cooling jacket circuit was still run with the last temperature set point. After further 60 minutes, methanol stream was stopped. The hot oil was circulating at the last set point until 2 h and interrupted just before the plant depressurization and the start with nitrogen purging. The reactor was slowly depressurized to remove the residual propylene in the vent line and was flushed with nitrogen (N₂ purge, gas flow 100 NI/h) for two hours to remove methanol and dry the catalyst. Total time for the intermediate shutdown were four hours.

Afterwards, the reactor was restarted and epoxidation of propylene was conducted in accordance with Reference Example 3 for three hours. After three hours, the reactor was finally shutdown following the procedure as described above for the intermediate shutdown. Total time for the final shutdown were also four hours.

The O₂ concentration in the vent stream at the outlet of the second separator was measured according to Reference Example 1 over the complete time of epoxidation, intermediate shutdown, epoxidation and final shutdown (“O₂ concentration after the second separator”). The results are graphically depicted in FIG. 1, wherein the O₂ concentration indicated in volume-% based on the total volume of the vent stream on the y-axis is shown versus the time in hours on the x-axis of the intermediate shutdown and during the epoxidation phase between intermediate and final shutdown. “0 h” on the x-axis indicated the point in time after 600 hours of epoxidation and directly at the stop of the feed of propylene. The volume-% value indicated at 0 h (0.8 volume-%) represents the O₂ content in the vent stream over the complete time of epoxidation, i.e. during a normal run.

### SUMMARY

Table 1 shows a summary of the sequence of steps and the composition of the combined feed stream to the reactor in Example 1 and Comparative Example 1 and the amount of oxygen measured (indicated in volume-% based on the total volume of the gaseous vent stream) in the vent line at the beginning and at the end of each step.

TABLE 1
Sequence of steps in Example 1 and Comparative Example 1

| | | | | | Comments O₂ |
|---|---|---|---|---|---|
| | Sequence of steps | | O₂ (volume-%) | | concentration in |
| Time | | Comparative | Example | Comparative | vent stream after |
| (h) | Example 1 | Example 1 | 1 | Example 1 | second separator |
| 0 | hydrogen | propylene stop | 0.4 | 0.8 | |
| | peroxide | | | | |
| | stop* | | | | |
| 0-1 | Methanol + | Methanol + | 0.4 | 0.7-0.8 | Example 1: constant |
| | propylene | hydrogen | | | Comparative Example |
| | | peroxide + aqueous | | | 1: constant |
| | | K₂HPO₄ buffer | | | |
| 1 | propylene | hydrogen | 0.4 | 0.7 | |
| | stop | peroxide stop* | | | |
| 1-2 | Methanol | Methanol | 0.4-0.2 | 0.7-0.8 | Example 1: 0.2% |
| | | | | | decrease |
| | | | | | Comparative Example |
| | | | | | 1: constant |
| 2 | Methanol | Methanol | 0.2 | 0.75 | |
| | stop | stop | | | |
| 2-4 | N₂ purge | N₂ purge | 0.2-0.0 | 0.8-0.0 | Example 1: decrease to |
| | | | | (peak | 0% in few min |
| | | | | at 2.25) | Comparative Example |
| | | | | | 1: gradual increase up |
| | | | | | to 2.25% followed by a |
| | | | | | decrease to 0% |
| 4-7 | Restart | Restart | 0.0-0.2 | 0.0-0.6 | Example 1: increase to |
| | | | | (peak | 0.2% in few minutes |
| | | | | at 1.25) | and then stable |
| | | | | | Comparative Example |
| | | | | | 1: gradual increase up |
| | | | | | to a maximum of 1.25% |
| | | | | | followed by a decrease |
| | | | | | to 0.5-0.6% |

*shortly afterwards, also stop of aqueous K₂HPO₄ stream

The initial oxygen concentration before starting the shutdown was slightly higher in Comparative Example 1 (0.8 volume-%) compared to Example 1 (0.4 volume-%), but both values were within the range of normal deviation of +0.5 volume-% observed in the mini plant, so that the results of Example 1 and Comparative Example 1 were directly comparable.

The results obtained, which are graphically depicted in FIG. 1, show that terminating the propylene feed to the combined feed stream first, while maintaining the feed of aqueous hydrogen peroxide solution to the static-mixer, i.e. using a combined stream comprising aqueous hydrogen peroxide solution and methanol as in Comparative Example 1 resulted in a significant increase of oxygen formation in the vent line; during the N₂ purging, the O₂ content reached a value which was more than 200% of the O₂ content in the normal run, i.e. a maximum of about 2.2 volume % was measured during N₂ purging compared to 0.8 volume-% for the normal run, and the O₂ content reached a value of more than 150% during the restart, i.e. a maximum of about 1.25 volume-% was measured during the restart compared to 0.8 volume-% for the normal run. Such an effect in turn resulted in issues in the propylene recovery section of any HPPO plant and also causes safety concerns. On the other hand, terminating first the feed of aqueous hydrogen peroxide to the static mixer, i.e. using a combined stream comprising propylene and methanol as in Example 1 clearly resulted in much lower oxygen concentration in the vent stream over the complete shutdown phase and also during epoxidation after restart. It could be seen that during the N₂ purging, the O₂ content reached a value which was more not more than 50% higher than the O₂ content in the normal run; in fact the O₂ value decreased, i.e. a minimum of about 0 volume-% was measured during N₂ purging compared to 0.4 volume-% for the normal run, and the O₂ content reached a value of less than 100% during the restart, i.e. a minimum of about 0.25 volume-% was measured during the restart compared to 0.4 volume-% for the normal run. Thus, the shutdown procedure of Example 1 with first stopping the feed of aqueous hydrogen peroxide was clearly advantageous.

### SHORT DESCRIPTION OF THE FIGURE

FIG. 1 shows the O₂ concentration in the vent stream (at the outlet of the second separator) measured according to Reference Example 1 over the complete time of epoxidation, intermediate shutdown, epoxidation and final shutdown for Example 1 and Comparative Example 1, wherein the O₂ concentration indicated in volume-% based on the total volume of the vent stream on the y-axis is shown versus the time in hours on the x-axis of the intermediate shutdown and during the epoxidation phase between intermediate and final shutdown. “0 h” on the x-axis indicated the point in time after 600 hours of epoxidation and directly at the stop of the feed of aqueous hydrogen peroxide solution.

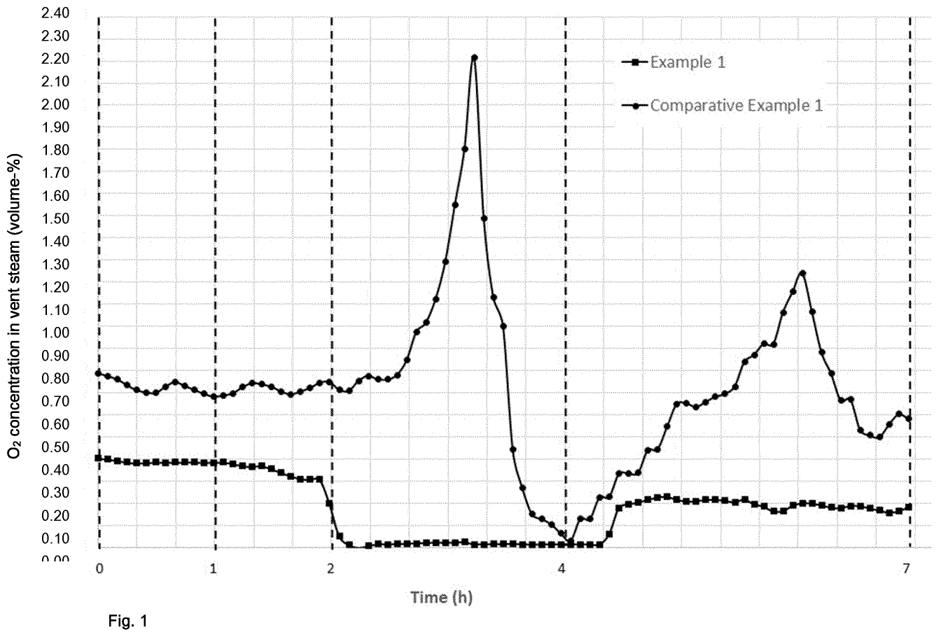

### Cited Literature

1.-15. (canceled)

16. A shutdown method for a process for preparing an olefin oxide comprising a normal run stage, wherein the normal run stage comprises providing olefin, hydrogen peroxide and organic solvent into an epoxidation zone comprising an heterogeneous epoxidation catalyst, so that a reaction mixture comprising olefin, hydrogen peroxide and organic solvent is formed and subjecting the reaction mixture to epoxidation reaction conditions in the epoxidation zone, thereby obtaining a mixture comprising olefin oxide and organic solvent;
wherein the shutdown method comprises
(a) providing olefin and organic solvent for a period of time t₁ to the epoxidation zone, so that a mixture is formed, which comprises less than 0.2 weight-% of hydrogen peroxide based on the total weight of the mixture;
(b) contacting the mixture from (a) over t₁ under epoxidation reaction conditions in the epoxidation zone with the heterogeneous epoxidation catalyst;
(c) removing during t₁ an effluent stream from the epoxidation zone, the effluent stream comprising olefin and organic solvent.

17. The shutdown method of claim 16, wherein the shutdown stage further comprises
(d) providing organic solvent for a period of time t₂ to the epoxidation zone, wherein the organic solvent comprises less than 0.2 weight-% of hydrogen peroxide and less than 1 weight-% of olefin, each based on the total weight of the organic solvent;
(e) contacting the organic solvent from (d) over t₂ under epoxidation reaction conditions in the epoxidation zone with the heterogeneous epoxidation catalyst,
(f) removing during t₂ an effluent stream from the epoxidation zone, the effluent stream comprising organic solvent.

18. The shutdown method of claim 16, wherein the shutdown stage further comprises
(g) providing a gaseous stream and passing said gaseous stream for a period of time t₃ into the epoxidation zone comprising the heterogeneous epoxidation catalyst and removing during t₃ the gaseous stream from the epoxidation zone.

19. The shutdown method of claim 16, wherein the gaseous stream used in (g) comprises at least 90 volume-% of nitrogen, based on the total volume of the gaseous stream.

20. The shutdown method of claim 16, wherein (a) comprises
(a.1) Providing olefin and organic solvent,
(a.2) combining olefin and organic solvent so that a mixture comprising olefin and organic solvent is formed, which comprises less than 0.2 weight-% of hydrogen peroxide based on the total weight of the mixture;
(a.3) feeding the mixture obtained in (a.2) for a period of time t₁ to the epoxidation zone.

21. The shutdown method of claim 16, wherein the effluent stream removed from the epoxidation zone during t₁ according to (b) and/or the effluent stream removed from the epoxidation zone during t₂ according to (d) and/or the gaseous stream removed from the epoxidation zone during t₃ according to (g), each optionally after an intermediate step, is introduced into a separation unit at a pressure in the range of from 0.05 to 0.5 MPa, thereby obtaining a gaseous stream comprising oxygen.

22. The shutdown method of claim 21, wherein in the intermediate step, the pressure is reduced from the range of from 0.5 to 5.0 MPa to a pressure in the range of from 0.05 to 0.5 MPa.

23. The shutdown method of claim 16, wherein in (a) an additive is provided to the epoxidation zone so that the mixture formed in (a) comprises an additive.

24. The shutdown method of claim 23, wherein providing the additive in (a) is stopped before expiry of t₁, and wherein no additive is added during (d) and/or during (g).

25. The shutdown method of claim 16, wherein the heterogeneous epoxidation catalyst comprises a zeolitic material having a framework structure comprising Si, O and Ti.

26. The shutdown method of claim 16, wherein the organic solvent is an organic epoxidation solvent.

27. The shutdown method of claim 16, wherein the olefin is a C2-C10 alkene.

28. A process for preparing an olefin oxide comprising a normal run stage and a shutdown stage, wherein the normal run stage comprises
(A) providing olefin, hydrogen peroxide, water and organic solvent into an epoxidation zone comprising an heterogeneous epoxidation catalyst, so that a reaction mixture comprising olefin, hydrogen peroxide, water and organic solvent is formed;
(B) subjecting the reaction mixture from (I) to epoxidation reaction conditions in the epoxidation zone, thereby obtaining a mixture comprising olefin oxide, water and organic solvent;
(C) removing an effluent stream from the epoxidation zone, comprising olefin oxide, water and organic solvent;
wherein the shutdown stage comprises
(a) providing olefin and organic solvent for a period of time t₁ to the epoxidation zone, so that a mixture is formed, which comprises less than 0.2 weight-% of hydrogen peroxide based on the total weight of the mixture;
(b) contacting the mixture from (a) over t₁ under epoxidation reaction conditions in the epoxidation zone with the heterogeneous epoxidation catalyst;
(c) removing during t₁ an effluent stream from the epoxidation zone, the effluent stream comprising olefin, optionally olefin oxide and organic solvent.

29. The process for preparing an olefin oxide according to claim 28, wherein the shutdown stage further comprises
(d) providing organic solvent for a period of time t₂ to the epoxidation zone, wherein the organic solvent comprises less than 0.2 weight-% of hydrogen peroxide and less than 1 weight-% of olefin, each based on the total weight of the organic solvent;
(e) contacting the organic solvent from (d) over t₂ under epoxidation reaction conditions in the epoxidation zone with the heterogeneous epoxidation catalyst,
(f) removing during t₂ an effluent stream from the epoxidation zone, the effluent stream comprising organic solvent.

30. The process for preparing an olefin oxide according to claim 28, wherein the shutdown stage further comprises
(g) providing a gaseous stream and passing said gaseous stream for a period of time t₃ into the epoxidation zone comprising the heterogeneous epoxidation catalyst and removing during t₃ the gaseous stream from the epoxidation zone.
